(19) United States
(12) Patent Application Publication
(10) Pub. No.: US 2021/0097895 A1
(43) Pub. Date: Apr. 1, 2021

(54) ALERT SLEEVE

(71) Applicant: Artemax, Inc., Brookfield, WI (US)

(72) Inventor: Bryan Waltersdorf, Oconomowoc, WI (US)

(21) Appl. No.: 17/032,201

(22) Filed: Sep. 25, 2020

Related U.S. Application Data
(60) Provisional application No. 62/906,214, filed on Sep. 26, 2019.

(51) Int. Cl. G09F 3/00; A61B 90/92; A61B 90/94

(52) U.S. Cl. CPC G09F 3/005; A61B 90/94; A61B 90/92

(57) ABSTRACT

An alert sleeve is configured to be arranged on a wristband for a person's wrist. The wristband includes a first end and a second end configured to be coupled to the first end to complete the wristband around the person's wrist. The alert sleeve includes a sleeve portion including an outer surface and an inner surface. The alert sleeve includes a passage defined through the sleeve portion, such that the inner surface is in facing relationship with the passage. The alert sleeve includes an identification characteristic on the sleeve portion. The alert sleeve is configured to be arranged on the wristband by moving the first end of the wristband through the passage of the alert sleeve and subsequently coupling the second end to the first end.

### CROSS-REFERENCE TO RELATED APPLICATIONS

This application claims priority to co-pending U.S. Provisional Patent Application No. 62/906,214 filed on Sep. 26, 2019, the entire content of which is incorporated herein by reference.

### FIELD OF THE INVENTION

The present invention relates to identification systems, and more particularly to attachments to wristbands for identification systems.

### BACKGROUND OF THE INVENTION

Patients in hospitals wear wristbands to help identify their patient. Attachments are coupled to the wristbands to identify further characteristics about that person, such as certain medical conditions.

### SUMMARY OF THE INVENTION

The present invention provides, in one aspect, an alert sleeve configured to be arranged on a wristband for the person's wrist. The wristband includes a first end and a second end configured to be coupled to the first end to complete the wristband around the person's wrist. The alert sleeve comprises a sleeve portion including an outer surface and an inner surface, a passage defined through the sleeve portion, such that the inner surface is in facing relationship with the passage, and an identification characteristic on the sleeve portion. The alert sleeve is configured to be arranged on the wristband by moving the first end of the wristband through the passage of the alert sleeve and subsequently coupling the second end to the first end.

The present invention provides, in another aspect, an identification assembly for a person. The identification assembly comprises a wristband for the person's wrist. The wristband includes a first end and an opposite second end configured to be coupled to the first end to complete the wristband around the person's wrist. The identification assembly further comprises an alert sleeve including a sleeve portion including an outer surface and an inner surface, a passage defined through the sleeve portion, such that the inner surface is in facing relationship with the passage, and an identification characteristic on the sleeve portion. The alert sleeve is configured to be arranged on the wristband by moving the first end of the wristband through the passage of the alert sleeve and subsequently coupling the second end to the first end.

The present invention provides, in yet another aspect, an alert sleeve configured to be arranged on a wristband for a person's wrist. The alert sleeve comprises a first end, a second end opposite the first end, and an identification characteristic. The alert sleeve is configured to be arranged on the wristband by wrapping the first end around the wristband and coupling the first end to the second end.

The present invention provides, in yet another aspect, an identification assembly for a person. The identification assembly comprises a wristband for the person's wrist, and an alert sleeve including first end, a second end opposite the first end, and an identification characteristic. The alert sleeve is configured to be arranged on the wristband by wrapping the first end around the wristband and coupling the first end to the second end.

The present invention provides, in yet another aspect, a method of coupling an alert sleeve with an identification characteristic to a wristband for a person. The method comprises moving a first end of the wristband through a passage of the alert sleeve and coupling the first end of the wristband to a second end of the wristband to complete the wristband around the person's wrist.

The present invention provides, in yet another aspect, a method of coupling an alert sleeve with an identification characteristic to a wristband for a person. The method comprises wrapping a first end of the alert sleeve around the wristband and coupling the first end to an opposite second end of the alert sleeve.

Other features and aspects of the invention will become apparent by consideration of the following detailed description and accompanying drawings.

### BRIEF DESCRIPTION OF THE DRAWINGS

FIG. 1 is a perspective view of an identification assembly including a wristband and an alert sleeve.

FIG. 2 is a perspective view of the identification assembly of FIG. 1, with the alert sleeve arranged on the wristband.

FIG. 3 is a perspective view of another embodiment of an alert sleeve having a removable portion.

FIG. 4 is a perspective view of the alert sleeve of FIG. 3, with the removable portion removed and an adhesive portion exposed.

FIG. 5 is a plan view of another embodiment of an alert sleeve having a removable portion.

FIG. 6 is a plan view of the alert sleeve of FIG. 5, with the removable portion removed and an adhesive portion exposed.

FIG. 7 is a perspective view of the wristband of FIG. 5 ready to be wrapped around a wrist.

FIG. 8 is a perspective view of the wristband of FIG. 5 partially wrapped around a wrist.

FIG. 9 is a perspective view of the wristband of FIG. 5 wrapped around a wrist.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

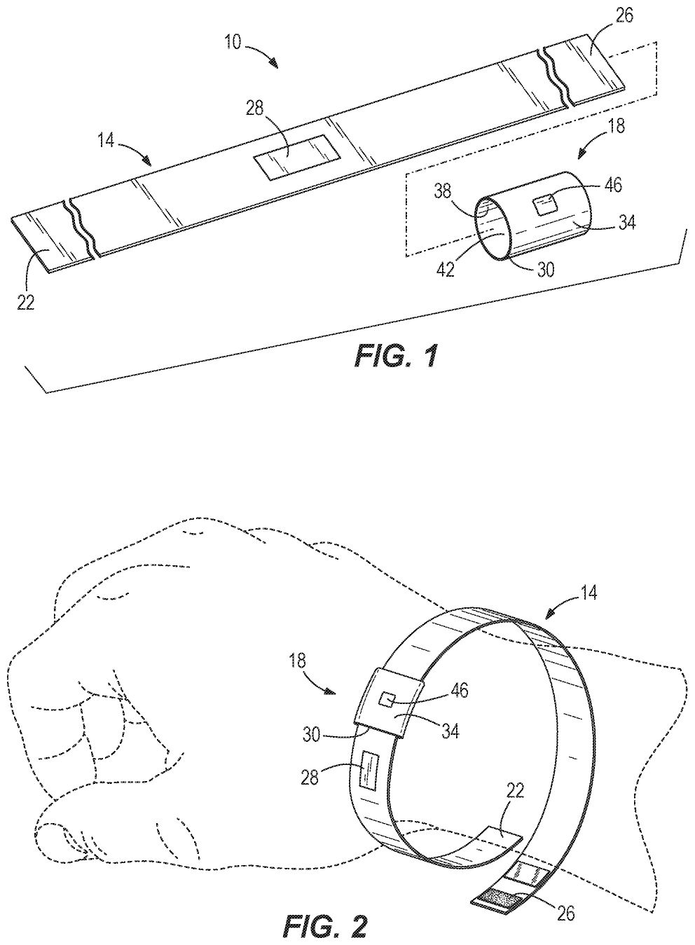

An identification assembly 10 is shown in FIG. 1 and includes a wristband 14 and an alert sleeve 18 configured to be arranged on the wristband 14. The wristband 14 includes a first end 22 and an opposite second end 26 configured to be coupled to the first end 22 in order to complete the wristband around a person's wrist. The wristband may also include identification information 28 about the person wearing the wristband, such as the person's name or other basic information. The alert sleeve 18 includes a sleeve portion 30 having an outer surface 34 and an inner surface 38. A passage 42 is defined through the sleeve portion 30. In some embodiments the alert sleeve 18 is formed of a flexible film material, such as Polyvinyl Chloride, Polyethylene, or Polyester. Thus, the alert sleeve 18 is comfortable, and does not irritate the person's wrist over a period of time in which the wristband and alert sleeve 18 rub against the person's wrist. This is particularly advantageous when the person is geriatric or has sensitive skin. In some embodiments, the alert sleeve 18 is opaque and in other embodiments, the alert sleeve 18 is clear or translucent.

An alert characteristic 46 is arranged on the outer surface 34 of the sleeve portion 30 of the alert sleeve 18. In some embodiments, the alert characteristic 46 can be a color, one or more alphanumeric characters, a symbol, or a combination thereof. The alert characteristic 46 provides information about the person wearing the wristband 14 that might not be apparent from the wristband 14 itself. For instance, in a hospital, where the identification information 28 on the wristband 14 indicates the name of the wearer, the alert characteristic 46 may be an alert and read “ALLERGY”, “LATEX ALLERGY”, “FALL RISK”, “RESTRICTED EXTREMITY”, or “DNR” (Do Not Resuscitate). In addition, the alert characteristic 46 and/or the alert sleeve 18 itself may be color-coded red with the “ALLERGY” alert characteristic 46, green with the “LATEX ALLERGY” alert characteristic 46, yellow with the “FALL RISK” alert characteristic, pink with the “RESTRICTED EXTREMITY” alert characteristic 46, and blue with the “DNR” (Do Not Resuscitate) alert characteristic 46. At a concert venue, where the wristband 14 indicates the wearer is entitled lawful entry, the alert characteristic 46 of the alert sleeve may indicate that the wearer is additionally of legal age to consume alcohol, or is a “VIP” (Very Important Person) entitled to backstage entrance, seating sections or special access.

In operation, the alert sleeve 18 is arranged on the wristband 14 by sliding the first end 22 of the wristband 14 through the passage 42 of the alert sleeve 18 and then coupling the first end 22 to the second end 26 of the wristband around the person's wrist, as shown in FIG. 2. In addition to the identification information 28 on the wristband 14 itself, the alert characteristic 46 on the outer surface 34 of the sleeve portion 30 of the alert sleeve 18 can be used to quickly identify an additional characteristic about the wearer of the wristband 14.

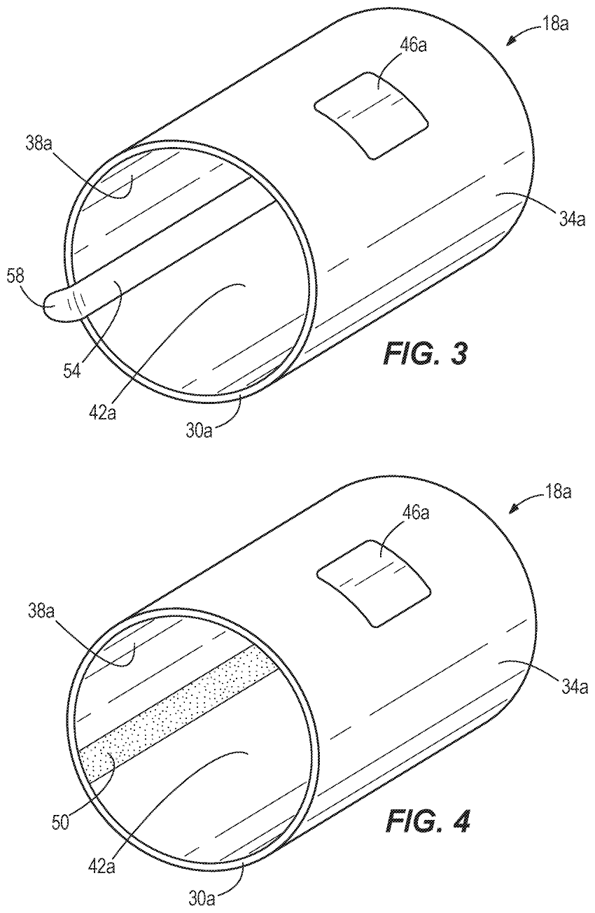

FIGS. 3 and 4 show another embodiment of an alert sleeve 18a, which is the same as the alert sleeve 18 of FIGS. 1 and 2 with like parts adding an “a”, and the following differences and additions explained below. In the alert sleeve 18a, the inner surface 38a of the sleeve portion 30a includes an adhesive portion 50 (FIG. 4) that is covered by a removable portion 54 (FIG. 3). In some embodiments, the removable portion 54 includes a tab 58 extending from the sleeve portion 30a, such that a person can grasp the tab 58 to remove the removable portion 54 and thereby expose the adhesive portion 50. In some embodiments, there is more than one adhesive portions 50 respectively covered by removable portions 54.

In operation, the alert sleeve 18a is arranged on the wristband 14 by sliding the first end 22 of the wristband 14 through the passage 42a of the alert sleeve 18a and then coupling the first end 22 to the second end 26 of the wristband 14. Then the person can grasp the tab 58 to remove the removable portion 54 and thereby expose the adhesive portion 50. The person can then flatten the alert sleeve 18a, causing the passage 42a to collapse and the adhesive portion 50 to become adhered to the wristband 14, such that the alert sleeve 18a is no longer able to translate along or be removed from the wristband 14. If desired, the alert sleeve 18a can be secured to the wristband 14 prior to securing the wristband into a loop on the person's wrist. Because the alert sleeve 18a can be secured to the wristband without the need of tools, the alert sleeve 18a is easy to use.

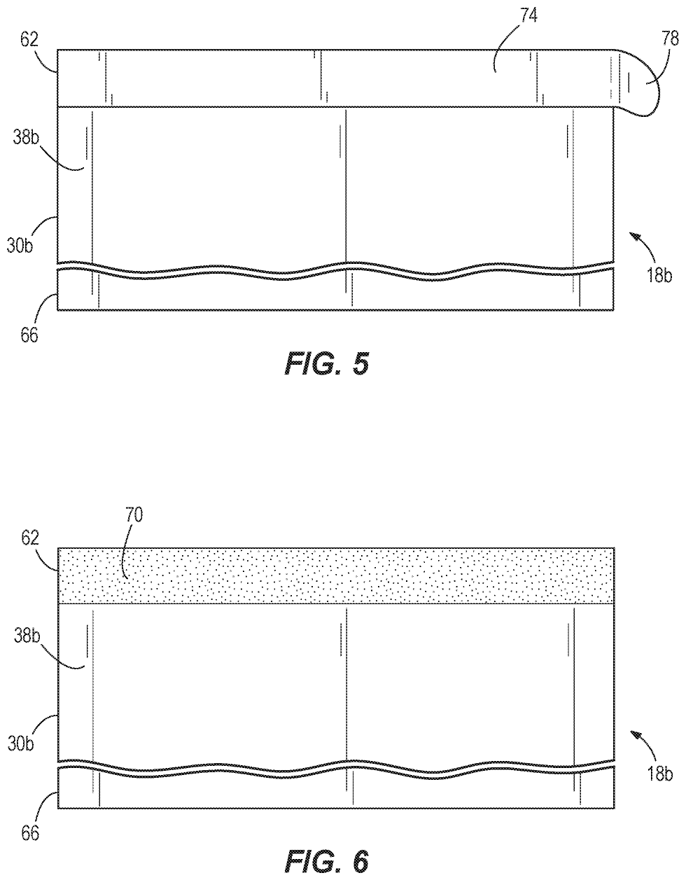

FIGS. 5-9 show another embodiment of an alert sleeve 18b, which is the same as the alert sleeve 18 of FIGS. 1 and 2 with like parts adding a “b”, and the following differences and additions explained below. The alert sleeve 18b is not pre-arranged as a tubular sleeve, as with the alert sleeve 18 of FIGS. 1-2 and the alert sleeve 18a of FIGS. 3 and 4. Rather, the sleeve portion 30b of the alert sleeve 18b is arranged as a strip with a first end 62 and a second end 66 that is opposite the first end 62. On the first end 62, the inner surface 38b of the sleeve portion 30b includes an adhesive portion 70 (FIG. 6) that is covered by a removable portion 74 (FIG. 5). In some embodiments, the removable portion 74 includes a tab 78 extending from the sleeve portion 30b, such that a person can grasp the tab 78 to remove the removable portion 74 and thereby expose the adhesive portion 70.

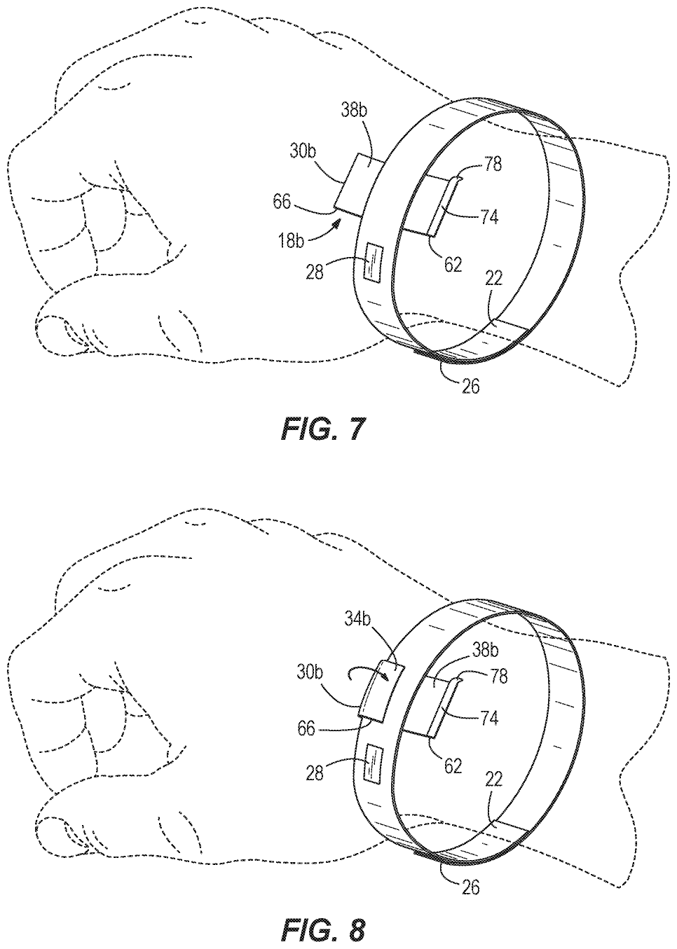

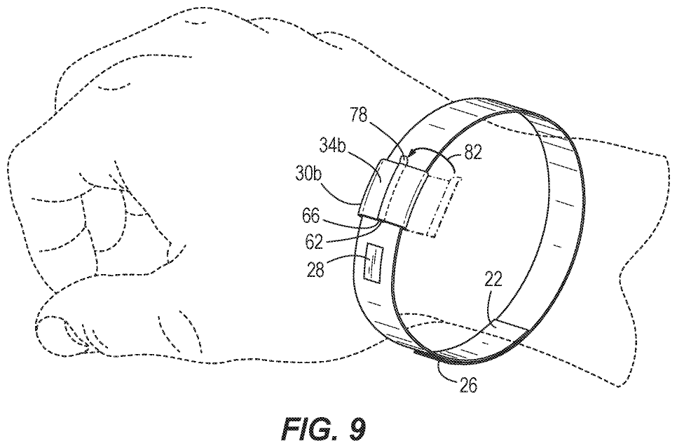

In operation, the first end 22 of the wristband 14 does not need to be slid through the passage 42b of the alert sleeve 18b in order to couple the alert sleeve 18b to the wristband 14. Rather, the first end 22 may already be coupled to the second end 26 of the wristband 14 on the person's wrist, as shown in FIGS. 7-9. Then, the person can slide the alert sleeve 18b under the wristband 14 (FIG. 7) and fold the second end 66 over the wristband 14 (FIG. 8). Next the person can fold the first end 62 of the alert sleeve 18b around the wristband 14, as indicated by arrow 82, until the first end 62 is arranged proximate the second end 66 of the wristband 14 (FIG. 9). Then the person can grasp the tab 78 to remove the removable portion 74 and thereby expose the adhesive portion 70. The person can then flatten the alert sleeve 18b, causing the passage 42b to collapse and the adhesive portion 70 to become adhered to the second end 66 or both the second end 66 and the wristband 14, such that the alert sleeve 18b is no longer able to translate along or be removed from the wristband 14. As can be appreciated, this embodiment of the alert sleeve is useful if it is desired to add an alert sleeve 18b after the wristband 14 is secured into a loop on the person's wrist.

Various features of the invention are set forth in the following claims.

1. An alert sleeve configured to be arranged on a wristband for a person's wrist, the wristband including a first end and a second end configured to be coupled to the first end to complete the wristband around the person's wrist, the alert sleeve comprising:
a sleeve portion including an outer surface and an inner surface;
a passage defined through the sleeve portion, such that the inner surface is in facing relationship with the passage; and
an identification characteristic on the sleeve portion,
wherein the alert sleeve is configured to be arranged on the wristband by moving the first end of the wristband through the passage of the alert sleeve and subsequently coupling the second end to the first end.

2. The alert sleeve of claim 1, wherein the alert sleeve includes an adhesive portion on the inner surface and a removable portion on the inner surface and covering the adhesive portion, such that after the alert sleeve has been arranged on the wristband, the removable portion is configured to be removed from the inner surface to expose the adhesive portion, such that the adhesive portion can engage against the wristband to adhere the alert sleeve to the wristband.

3. The alert sleeve of claim 1, wherein the identification characteristic is on the outer surface.

4. The alert sleeve of claim 3, wherein the identification characteristic includes a color.

5. The alert sleeve of claim 3, wherein the identification characteristic includes alphanumeric characters.

6. The alert sleeve of claim 1, wherein the alert sleeve is formed of a flexible material.

7. An identification assembly for a person, the identification assembly comprising:
a wristband for the person's wrist, the wristband including a first end and an opposite second end configured to be coupled to the first end to complete the wristband around the person's wrist; and
an alert sleeve including
a sleeve portion including an outer surface and an inner surface,
a passage defined through the sleeve portion, such that the inner surface is in facing relationship with the passage,
an identification characteristic on the sleeve portion,
wherein the alert sleeve is configured to be arranged on the wristband by moving the first end of the wristband through the passage of the alert sleeve and subsequently coupling the second end to the first end.

8. The identification assembly of claim 7, wherein the alert sleeve includes an adhesive portion on the inner surface and a removable portion on the inner surface and covering the adhesive portion, such that after the alert sleeve has been arranged on the wristband, the removable portion is configured to be removed from the inner surface to expose the adhesive portion, such that the adhesive portion can engage against the wristband to adhere the alert sleeve to the wristband.

9. The identification assembly of claim 7, wherein the identification characteristic is on the outer surface.

10. An alert sleeve configured to be arranged on a wristband for a person's wrist, the alert sleeve comprising:
a first end;
a second end opposite the first end; and
an identification characteristic,
wherein the alert sleeve is configured to be arranged on the wristband by wrapping the first end around the wristband and coupling the first end to the second end.

11. The alert sleeve of claim 10, wherein the first end includes an adhesive portion and a removable portion covering the adhesive portion, such that after the first end has been wrapped around the wristband, the removable portion is removable from the first end to expose the adhesive portion, such that the adhesive portion can adhere the first end to the second end.

12. The alert sleeve of claim 11, wherein the removable portion is removable from the first end to expose the adhesive portion, such that the adhesive portion can adhere the first end to the second end and the wristband.

13.-20. (canceled)
